(19) United States
(12) Patent Application Publication
(10) Pub. No.: US 2007/0148770 A1
(43) Pub. Date: Jun. 28, 2007

(54) MACROPOROUS CHITOSAN BEADS AND PREPARATION METHOD THEREOF

(75) Inventors: Seo Young JEONG, Kyunggi-do (KR); Eunhee BAE, Seoul (KR); Ick Chan KWON, Seoul (KR); Kuiwon CHOI, Seoul (KR)

Correspondence Address: JHK LAW, P.O. BOX 1078, LA CANADA, CA, 91012-1078, US

(73) Assignee: Korea Institute of Science and Technology, Seoul (KR)

(21) Appl. No.: 11/565,541

(22) Filed: Nov. 30, 2006

Related U.S. Application Data
(62) Division of application No. 10/168,701, filed on Jun. 17, 2002, now abandoned.

(51) Int. Cl. C12N 5/08; C12N 5/00

(52) U.S. Cl. 435/325; 435/404; 435/366

(57) ABSTRACT

The present invention relates to macroporous chitosan beads having 5-200 μm in size of relatively large and uniform pores that are distributed from surface to core region. The macroporous chitosan beads of the present invention make cell culturing efficient. Cells can attach to them efficiently due to their large surface area.

Thus far, many naturally occurring polymers and synthetic polymers have been used as matrices for cell culture. For example, PGA (polyglycolic acid) mesh was used to make three-dimensional, porous bone substitutes which allow many cells to adhere thereto, in addition to supporting fast tissue regeneration and being superior in biodegradability (Vunjak-Nonakovi, G. et al., Journal of Biotechnology Progress, Vol. 14, 193-202, 1998). Du, C. et al. synthesized nHAC (nano-HAp/collagen) (Journal of Biomedical Materials Research, Vol. 44, 407-415, 1999). PLLA (poly-L-lactic acid) was successfully used to culture osteoblasts (Lo et al., Journal of Biomedical Materials Research, Vol. 30, 475-484, 1996; Evans, G R. et al., Journal of Biomaterials, Vol. 20, 1109-1115, 1999). PGA and PLLA were formed into meshes, or three-dimensional porous scaffolds using a solvent-casting particulate-leaching method, onto which chondrocytes are grown (Freed et al., Journal of Biomedical Materials Research Vol. 27, 11-23, 1993). There was made an attempt to culture fibroblast cells on a porous matrix prepared from PEG (polyethylene glycol) conjugated with fibrinogen (Pandit, A. S. et al., Journal of Biomaterials Application, vol. 12, 222-236). Another success in culturing fibroblasts was achieved by using tubular PGA formed by a spray-casting method of PLLA or PLGA (poly-D,L-lactic-co-glycolic acid) solution in chloroform, which showed increased compressive strength.

For effective cell culture, fundamentally, porous matrices are required to allow as many cells to adhere thereto as possible in a limited space easily and evenly, as well as facilitating the growth of cells. However, the above matrices cannot meet the requirements satisfactorily.

Proposed to compensate for the deficiencies of the above matrices were bead-like matrices. A research on various bio-compatible materials with growth factor properties effective for hemostasis in case of trauma resulted in the finding that positively charged beads were much more effective for stopping hemorrhage (Wu, L. et al., Journal of Surgical Research, vol. 85, 43-50, 1999). Alginate beads were used to culture chondrocytes and, after 1-2 days of cell culturing, IL-1β (interleukin-1β) was added to facilitate the formation of extracellular matrix (Beekman, B. et al., Osteoarthritis Cartilage, Vol. 5, 330-340, 1998).

In addition, other porous matrices for cell culture have been developed from gelatin, collagen, hyaluronic acid, cellulose and glass. Porous gelatin beads are polymerized by addition of HEMA (2-hydroxyethyl methacrylate) and EDM (ethylene glycol dimethacrylate) and made to be porous by repeated cycles of freezing and thawing.

Such gelatin beads enable various kinds of cells to be attached thereto. Thereafter, the cells are implanted to tissues in order to study tissue substitutions. The beads can be varied in size depending on materials, but are not suitable for use in cell culture owing to their small pore sizes ranging from 0.7 to 2.6 μm. Bead matrices enjoy advantages of accommodating a large number of cells within a limited space, enabling the cells to grow well, and efficiently releasing products. However, bead matrices made of alginate or gelatin have difficulty in forming pores of desired sizes and in allowing uniform distributions thereon and therein. When being made of collagen or glass, beads suffer from being poor in biocompatibility. Therefore, these beads are unsuitable as matrices for cell adsorption in terms of cell versatility and adsorption strength.

For effective use in study on tissue substitution through cell implantation, polymers are required to have ability of cell attachment and be of biocompatibility, biodegradability, plasticity, and porosity. Superior as they are in plasticity for size and shape to natural polymers, synthetic polymers are poorer in biocompatibility and biodegradability. Therefore, synthetic polymers are apt to cause various side effects upon direct tissue implantation. For these reasons, naturally occurring polymers which are safe and have a variety of utilities are under active study.

Chitin, a precursor of chitosan, is quantitatively found in the shells of crustaceans, such as crabs and shrimps, and insects, and in the cell walls of fungi, mushrooms and bacteria. It is a polymer consisting of N-acetyl-D-glucosamine repeating units which are linked to each other via a (1→4)-β-glycosidic linkage. Chitosan, an alkaline polysaccharide prepared by N-deacetylating chitin with a high concentration of alkali, is known to be superior in ability of cell attachment, biocompatibility, biodegradability, and plasticity to the above-mentioned synthetic polymers.

Thanks to these advantages, many attempts have been made to utilize chitosan as a matrix for cell culture. For example, glutaraldehyde-crosslinked chitosan and fructose-modified chitosan were utilized as matrices for culturing hepatocytes (Yagi, et al., Biological Pharmaceutical Bulletin, Vol. 20, No. 6, 708-710 & Vol. 20, No. 12, 1290-1294, 1997). These chitosan matrices can be prepared by mixing glutaraldehyde or fructose with pure chitosan to increase cell attachment and formed into desired shapes. However, the cell culture using these modified chitosan matrices cannot go beyond two-dimensional culturing system because cells are adsorbed only to the surfaces of the matrices.

Chitosan films with desired pore sizes were developed by various freeze-drying techniques and used in tissue engineering (Madihally, S. V. et al., Journal of Biomaterials, Vol. 20, 1133-1142, 1999). These chitosan films are very significant in terms of providing desired sizes of pores, but still remain limited to two-dimensional cell culturing techniques.

In addition, chitosan beads which were prepared through freeze-drying were reported (Tzu-Yang, et al., Journal of Industrial Engineering Chemical Research, Vol. 36, 3631-3638, 1997). The chitosan beads were modified by cross-linking glutaraldehyde to amino residues of chitosan beads and measured to show a high adsorption rate for cadmium ions.

Novel chitosan beads were also found in U.S. Pat. No. 5,864,025 issued to Wolfgang, G et al. on Jan. 26, 1999. They used non-magnetic succinic anhydride to give chitosan beads with carboxylic groups. They were reacted with ferrous chloride (FeCl₂) and washed with excess amount of water to afford magnetic chitosan beads which can be used to purify proteins or to absorb magnetic materials, as have been reported. Owing to their small pore sizes, the porous chitosan beads are used for the adsorption and/or purification of ions or magnetic materials. However, nowhere are found the use of the porous chitosan beads as matrices for cell culture.

### SUMMARY OF THE INVENTION

Based on its excellence in ability of cell attachment, biocompatibility, biodegradability and plasticity, chitosan was studied in order to prepare a macroporous bead with evenly distributed large pores in which cells can be cultured well. Leading to the present invention, the thorough and intensive research, conducted by the present inventors, resulted in the finding that a chitosan solution undergoes phase separation in an organic solvent, so that macroporous chitosan beads can be made to have uniform pores thereon and therein.

Therefore, it is an object of the present invention to overcome the problems encountered in prior arts and to provide macroporous chitosan beads which have uniform pores therein and thereon.

It is another object of the present invention to provide macroporous chitosan beads which have such large surface areas as to adsorb cells thereto.

It is a further object of the present invention to provide macroporous chitosan beads which are superior in ability of cell attachment, biocompatibility, and biodegradability and thus useful in cell growth, angiogenesis and nutrient diffusion.

It is still a further object of the present invention to provide a method for preparing macroporous chitosan beads.

It is still another object of the present invention to provide a method for culturing animal and plant cells using the macroporous chitosan beads.

### BRIEF DESCRIPTION OF THE DRAWINGS

FIG. 1 is a SEM photograph showing the surface of the porous chitosan beads of the present invention before cells are cultured on and in the beads.

FIG. 2 is a SEM photograph showing a cross section of the porous chitosan beads of the present invention before cells are cultured on and in the beads.

FIG. 3 is a SEM photograph showing the surface of the porous chitosan beads of the present invention after hepatocytes are cultured in and on the porous chitosan beads for 10 days.

### DETAILED DESCRIPTION OF TIE INVENTION

Before the present macroporous chitosan beads and preparation thereof are disclosed or described, it is to be understood that the terminology used therein is for the purpose of describing particular embodiments only and is not intended to be limiting.

The term “a chitosan solution” as used herein means an aqueous acetic acid solution containing chitosan. The term “an aqueous chitosan solution” as used herein means a solution of a water-soluble chitosan in deionized water.

Also, the term “chitosan beads” or “porous chitosan beads” as used herein means porous chitosan particles of 1-4 mm with relatively uniform pores, prepared from a chitosan solution, an aqueous chitosan solution or mixtures thereof.

Meanwhile, the term “a matrix” or “a matrix for cell culture” as used herein means a solid support or carrier to which cells are attached while being cultured in media so as to proliferate.

In one aspect, the present invention pertains to porous chitosan beads for cell culture, which are excellent in biocompatibility, biodegradability, ability of cell attachment and plasticity with pores being large and uniform in size. With these advantages, the porous chitosan beads are very useful matrices on which various kinds of animal and plant cells can be cultured. The porous chitosan beads of the present invention can be used as matrices for culturing all kinds of animal and plant cells and particularly useful for culturing hepatocytes, fibroblasts, osteoblasts, epithelial cells, and viral packaging cells.

As for the pores of the porous chitosan beads of the present invention, they are preferably in the range of 1-500 μ and more preferably in the range of 5-200 μm. The beads preferably range in size from 0.1 to 10 mm and more preferably from 1 to 4 mm.

In another aspect, the present invention pertains to a method for preparing porous chitosan beads. The preparation of chitosan beads starts with a chitosan solution, an aqueous chitosan or a mixture thereof. As mentioned above, the chitosan solution is prepared by dissolving chitosan in an aqueous acetic acid solution while the aqueous chitosan solution is prepared by dissolving water-soluble chitosan in deionized water. Next, the solution is added drop wise to an organic solvent of low temperature or liquid nitrogen to give beads. Finally, the chitosan beads are freeze-dried.

While chitosan is soluble in acid, water-soluble chitosan shows significant solubility in water. Useful in the present invention is the chitosan with an average molecular weight of within the range of 5,000-1,000,000. For dissolving chitosan, the acetic acid solution preferably has a concentration of 0.1-10% by weight. After completion of the dissolution, the chitosan is preferably present at an amount of 0.1-20% by weight in the acetic acid solution. When being dissolved in deionized water, the water-soluble chitosan preferably ranges in concentration from 0.5 to 1.5% by weight. Higher concentrations result in smaller pore sizes. Thus, when the concentration of the chitosan is higher than 4%, very small pores are formed, limiting the introduction and growth of cells.

When chitosan is used along with water-soluble chitosan, chitosan is preferably mixed at a weight ratio of 1:9-9:1 with the water-soluble chitosan. The higher the proportion of the water-soluble chitosan is, the greater the pore is in size.

Examples of the organic solvent useful in the present invention include chlorocyclohexane, chloropentane, n-hexane, dichloromethane, chloroform, and ethyl acetate. These organic solvents, having low melting points while not dissolving chitosan, are very useful in solidifying chitosan through phase separation due to difference in solubility and melting temperature. As seen from the examination for change in pore size depending on organic solvents, chloropentane makes pores larger than does dichloropentane.

It is preferred that the organic solvent is constantly maintained at low temperatures. If the temperature maintained constant is fluctuated, the solidified, porous beads suddenly melt at their surfaces to lose their porosity to the extent that the three-dimensional structure necessary for cell attachment and aiding cells to perform their functions is destroyed. The organic solvents are preferably maintained at −5 to −65° C. and liquid nitrogen at about −198° C. For example, lower temperatures lead to smaller pore sizes. On the other hand, if the organic solvents are maintained at too high temperatures, the phase separation due to temperature difference does not occur. The most preferable conditions for the present invention include the addition of a 1% chitosan solution to a chloropentane solvent maintained at −5 to −25° C. and the addition of a 1% aqueous chitosan solution to a chloroform solvent maintained at −5 to −25° C.

For maintaining the low temperatures of the organic solvents, dry ice or ethanol chilled by use of a freezer may be used. Alternatively, liquid nitrogen of about −198° C. may be used.

The porous chitosan beads thus prepared are homogeneous in size with a distribution ranging from 1 to 4 mm. To be useful as matrices for cell culture, the porous chitosan beads must be let to undergo various pre-treatments, for example, freeze-drying, neutralization to remove remaining acids and organic solvents, sterilization with ethanol, filling with culture media, and then, freeze-drying again.

In a further aspect, the present invention pertains to a method of culturing animal and plant cells using the porous chitosan beads. First, after the porous chitosan beads prepared are immersed in a culture medium, preculturing is conducted to attach cells to the porous chitosan beads. Following removal of unattached cells, the attached cells are proliferated while the old medium is changed with fresh medium. The preculturing for cell attachment is preferably conducted for 4-6 hours. It is preferred that the culture media are changed every two or three days.

Under various conditions concerning concentrations of chitosan and acetic acid and kinds and temperatures of organic solvents, porous chitosan beads were prepared, and used as matrices for culturing various kinds of cells, including hepatocytes, fibroblasts, osteoblasts, endothelial cells, and viral packaging cells.

As a result, various sizes of pores were formed according to preparation conditions. In detail, the pore size of the porous chitosan beads was found to become small as the organic solvents were maintained at lower temperatures or the chitosan solution or the aqueous chitosan solution is increased in concentration. That is, the pore size is determined by the temperature at which the phase separation of the chitosan solution or the aqueous chitosan solution occurs and by the concentration of the chitosan solution or the aqueous chitosan solution. Also, the kind of the organic solvent has influence on the determination of the pore size of the chitosan beads. When using chloropentane, the pore size was measured to be the largest. On the other hand, dichloropentane resulted in the smallest pores. In the case of mixtures of chitosan and water-soluble chitosan, the largest pores were obtained when a mixture of chitosan and water-soluble chitosan in the proportions of 4:6 were used. Comparison between chitosan and water-soluble chitosan at the same concentration leads to the conclusion that chitosan has an advantage over water-soluble chitosan in increasing the pore size.

In consequence, 1% aqueous chitosan and chloroform of −5 to −25° C. or 1% chitosan and chloropentane of −5 to −25° C. can bring about the largest size in the pores of the chitosan beads.

Over conventional matrices, the porous chitosan beads prepared by the method of the present invention show superiority in the adsorption of various kinds of animal and plant cells. Within 2-3 days after being adsorbed to the porous chitosan beads, the cells were grown into the inside of the beads as well as over the surfaces. Additionally, the hepatocytes cultured using the matrix of the present invention were found to maintain their cell functions as measured by various biochemical experiments.

### EXAMPLES

A better understanding of the present invention may be obtained in light of the following examples which are set forth to illustrate, but are not to be construed to limit the present invention.

### Example 1

### Preparation of Porous Beads Using 1% Chitosan Solution and Chloropentane

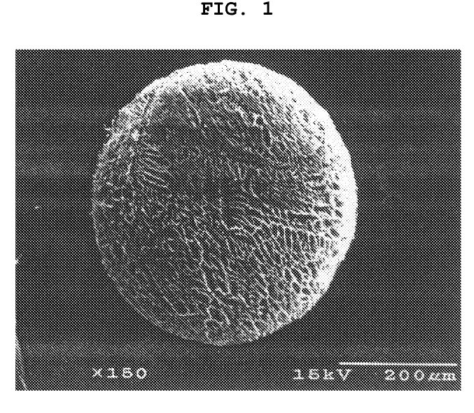

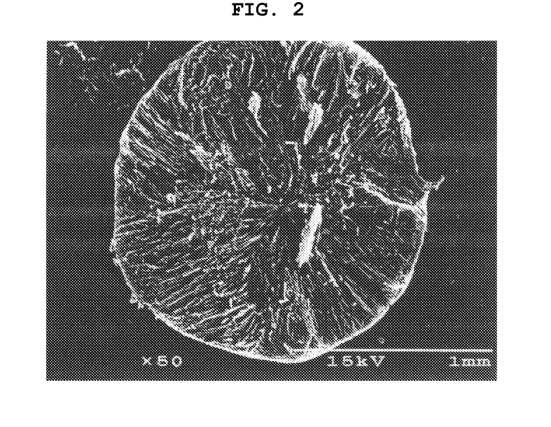

To 1% aqueous acetic acid solution was dissolved chitosan (Fluka, USA) at an amount of 1% by weight, after which the chitosan solution was slowly added to chloropentane (Sigma USA) maintained at −5 to −25° C., at −25 to −45° C., and at −45 to −65° C. by dry-ice containing ethanol (Sigma, USA), with the aid of a 10 ml syringe. The beads which were formed 5-10 sec after the addition, were separated by use of a spoon and frozen at −70° C. for 1 day, followed by freeze-drying for 2-3 days in a freeze-drier (Cole-Parmer Instrument Company, USA). They were observed for surface morphology under a scanning electron microscope and measured for pore size. The results are given in Table 1 below, and shown in FIGS. 1 and 2.

TABLE 1
Size changes of porous beads according to
the temperature change of organic solvent

| | Conc. Of | | Temp. of organic | |
|---|---|---|---|---|
| No. | chitosan (%) | Organic solvent | solvent (° C.) | Pore size (μm) |
| 1 | 1 | Chloropentane | −5˜−25 | 50˜150 |
| 2 | 1 | Chloropentane | −25˜−45 | 30˜100 |
| 3 | 1 | chloropentane | −45˜−65 | 10˜70 |

When porous chitosan beads were prepared using 1% chitosan under the same conditions of all parameters, except for the temperature of the organic solvent, as seen in Table 1, smaller pore sizes were obtained at lower temperatures.

### Example 2

### Preparation of Porous Beads Using 1% Chitosan and Various Organic Solvents

Chitosan beads were prepared in a manner similar to that of Example 1, except that a 1% aqueous acetic acid solution containing chitoson at an amount organic solvents such as chloropentane, n-hexane, dichloropentane, chloroform, and ethyl acetate, maintained at −5 to −25° C. were used.

The chitosan beads were observed with the aid of a scanning electron microscope and measured for pore size. The results are given in Table 2, below.

TABLE 2
Size changes of porous beads according to
the different organic solvent

| | Conc. Of | | Temp. of organic | Pore size |
|---|---|---|---|---|
| No. | chitosan (%) | Organic solvent | solvent (° C.) | (μm) |
| 1 | 1 | Chloropentane | −5˜−25 | 50˜150 |
| 4 | 1 | n-hexane | −5˜−25 | 20˜120 |
| 5 | 1 | Dichloropentane | −5˜−25 | 20˜100 |
| 6 | 1 | chloroform | −5˜−25 | 20˜80 |
| 7 | 1 | Ethyl acetate | −5˜−25 | 40˜100 |

Under the same conditions for all parameters, except for organic solvents, the average pore size of the chitosan beads were measured to be the smallest upon using chloroform and the greatest upon using chloropentane.

### Example 3

### Preparation of Porous Beads Using 2% Chitosan and Chloropentane

Chitosan beads were prepared in a manner similar to that of Example 1, except that a 1% acetic acid solutin containing chitosan at an amount of 2% by weight, and chloropentane maintained at −5 to −15° C. and −15 to −25° C. were used.

The chitosan beads were observed with the aid of a scanning electron microscope and measured for pore size. The changes in pore size with chitosan concentration are given in Table 3, below.

TABLE 3
Size changes of porous beads according to
the concentration of chitosan

| | Conc. Of | | Temp. of organic | |
|---|---|---|---|---|
| No. | chitosan (%) | Organic solvent | solvent (° C.) | Pore size (μm) |
| 1 | 1 | Chloropentane | −5˜−25 | 50˜150 |
| 8 | 2 | Chioropentane | −5˜−15 | 10˜100 |
| 9 | 2 | chloropentane | −15˜−25 | 10˜50 |

As apparent from Table 3, the chitosan beads have smaller average pore sizes as the concentration of the chitosan solution increases.

### Example 4

### Preparation of Porous Beads Using Solutions of 2% Chitosan in 1-4% Aqueous Acetic Acid and Chloropentane

Chitosan beads were prepared in a manner similar to that of Example 1, except that solutions of 2% (wt) chitosan in 1, 2, 3 and 4% aqueous acetic acid, and chloropentane maintained at −15 to −25° C. were used.

Observation under a scanning electron microscope revealed that the porous chitosan beads ranged in pore size from 10 to 80 μm. The observation results are given, along with the results of Example 3, in Table 4, below. As seen in Table 4, higher concentrations of the acetic acid solution resulted in larger pore sizes.

TABLE 4
Size changes of porous beads according to
the concentration of acetate

| | | | | Temp. of | |
|---|---|---|---|---|---|
| | Conc. Of | Conc. Of | Organic | organic | Pore size |
| No. | acetate (%) | chitosan (%) | solvent | solvent (° C.) | (μm) |
| 9 | 1 | 2 | Chloro- | −15˜−25 | 10˜50 |
| | | | pentane | | |
| 10 | 1˜4 | 2 | Chloro- | −15˜−25 | 30˜80 |
| | | | pentane | | |

### Example 5

### Preparation of Porous Beads Using 2% Chitosan and Liquid Nitrogen

Chitosan beads were prepared in a manner similar to that of Example 1, except that a solution of 2% (wt) chitosan in 1% aqueous acetic acid, and liquid nitrogen were used.

Observation under a scanning electron microscope revealed that the porous chitosan beads ranged in pore size from 5 to 50 μm. This observation agrees with the data obtained in Example 1, which led to the conclusion that lower temperatures make pore sizes smaller, because the temperatures of liquid nitrogen is much lower than those of organic solvents.

### Example 6

### Preparation of Porous Beads Using 2% Chitosan and Chlorocyclohexane

Chitosan beads were prepared in a manner similar to that of Example 1, except that a solution of 2% (wt) chitosan in 1% aqueous acetic acid, and chlorocyclohexane maintained at −5 to −15°C., −15 to −25° C., and −25 to −50° C. were used.

Observation under a scanning electron microscope revealed that the porous chitosan beads ranged in pore size from 10 to 150 μm. These observation results are given in Table 5, below.

TABLE 5
Size changes of porous beads according to the
temperature change of organic solvent in 2% chitosan.

| | Conc. Of | | Temp. of organic | |
|---|---|---|---|---|
| No. | chitosan (%) | Organic solvent | solvent (° C.) | Pore size (μm) |
| 12 | 2 | Chlorocyclo- | −5˜−15 | 50˜150 |
| | | hexane | | |
| 13 | 2 | Chlorocyclo- | −15˜−25 | 20˜80 |
| | | hexane | | |
| 14 | 2 | Chlorocyclo- | −25˜−50 | 10˜60 |
| | | hexane | | |

Under the same conditions for all parameters, except for organic solvent temperatures, as seen in Table 5, the average pore sizes of the chitosan beads were measured to be similar to those obtained upon using chloropentane, and to be smaller as the temperature decreases.

### Example 7

### Preparation of Porous Beads Using Mixtures of Chitosan and Water-Soluble Chitosan in Chloropentane

Chitosan beads were prepared in a manner similar to that of Example 1, except that solutions of 1% (wt) of mixtures of chitosan and water-soluble chitosan (Jakwang Co. Ltd., Korea) in the proportions of 8:2, 6:4, 4:6 and 2:8, and chloropentane maintained at −5 to −25° C. and −25 to −45° C. were used.

Observation under a scanning electron microscope revealed that the porous chitosan beads ranged in pore size from 10 to 120 μm. The changes in pore size according to proportions of the mixture and temperatures of the organic solvent are given in Table 6, below.

TABLE 6
Size changes of porous beads according to the
proportions of chitosan and water-soluble chitosan

| | chitosan:water- | | Temp. of organic | Pore |
|---|---|---|---|---|
| No. | soluble chitosan | Organic solvent | solvent (° C.) | size (μm) |
| 15 | 8:2 | Chloropentane | −5˜−25 | 10˜80 |
| 16 | 6:4 | Chloropentane | −5˜−25 | 20˜70 |
| 17 | 4:6 | Chloropentane | −5˜−25 | 30˜120 |
| 18 | 2:8 | Chloropentane | −5˜−25 | 20˜100 |
| 19 | 8:2 | Chloropentane | −25˜−45 | 10˜60 |
| 20 | 6:4 | Chloropentane | −25˜−45 | 20˜80 |
| 21 | 4:6 | Chloropentane | −25˜−45 | 20˜120 |
| 22 | 2:8 | Chloropentane | −25˜−45 | 20˜100 |

As seen in Table 6, higher proportions of the water-soluble chitosan made the pore size larger, while the temperature of the chloropentane had almost no influence on the pore size. Particularly using a mixture of 4:6 of chitosan and water-soluble chitosan, the chitosan beads showed the largest average pore size, which were measured to range from 30 to 120 μm.

### Example 8

### Preparation of Porous Beads Using Water-Soluble Chitosan in Chloropentane

Chitosan beads were prepared in a manner similar to that of Example 1, except that a tion of 1% (wt) of water-soluble chitosan in deionized water and chloropentane maintained at −5 to −25 °C., −25 to −45 ° C. and −45 to −65 ° C. were used.

Observation under a scanning electron microscope revealed that the porous chitosan beads ranged in pore size from 10 to 70 μm. A measurement was made of the pore sizes of the beads and the results are given in Table 7, below.

TABLE 7
Size changes of porous beads according to the temperature
change of organic solvent in water-soluble chitosan

| | Conc. Of | | Temp. of organic | |
|---|---|---|---|---|
| No. | chitosan (%) | Organic solvent | solvent (° C.) | Pore size (μm) |
| 23 | 1 | Chloropentane | −5˜−25 | 20˜60 |
| 24 | 1 | Chloropentane | −25˜−45 | 10˜70 |
| 25 | 1 | Chloropentane | −45˜−65 | 10˜60 |

As apparent from Table 7, the chitosan beads prepared from a water-soluble chitosan solution have pore sizes smaller than those of the chitosan beads prepared from a chitosan solution. Additionally, these chitosan beads did not undergo a great change in pore size according to temperatures, unlike the chitosan beads prepared from the chitosan solution.

### Example 9

### Preparation of Porous Beads Using Water-Soluble Chitosan and Various Organic Solvents

Chitosan beads were prepared in a manner similar to that of Example 1, except that a solution of 1% (wt) water-soluble chitosan in deionized water, and various organic solvents such as chloropentane, n-hexane, dichloropentane, chloroform, and ethyl acetate, maintained at −5 to −25° C. were used. Observation under a scanning electron microscope revealed that the porous chitosan beads ranged in pore size from 20 to 200 μm. When being prepared from water-soluble chitosan, the chitosan beads were measured for pore sizes according to kinds of organic solvents. The results are given in Table 8, below.

TABLE 8
Size changes of porous beads using water-soluble
chitosan according to the different organic solvent

| | Conc. Of | | Temp. of organic | Pore size |
|---|---|---|---|---|
| No. | chitosan (%) | Organic solvent | solvent (° C.) | (μm) |
| 23 | 1 | Chloropentane | −5˜−25 | 20˜60 |
| 26 | 1 | n-hexane | −5˜−25 | 30˜140 |
| 27 | 1 | Dichloro-methane | −5˜−25 | 30˜150 |
| 28 | 1 | chloroform | −5˜−25 | 50˜200 |
| 29 | 1 | ethylacetate | −5˜−25 | 40˜100 |

In the chitosan beads prepared from the water-soluble chitosan solution, as shown in Table 8, larger pore sizes were formed when using chloroform as an organic solvent than when using the other organic solvents. On the other hand, the chitosan beads prepared from the chitosan solution had the largest pore sizes upon using chloropentane (Table 2). From these results, it is apparent that the pore sizes of the chitosan beads prepared from the chitosan solution or the water-soluble chitosan solution are affected by kinds of organic solvents.

### Experimental Example 1

### Culture of Hepatocytes

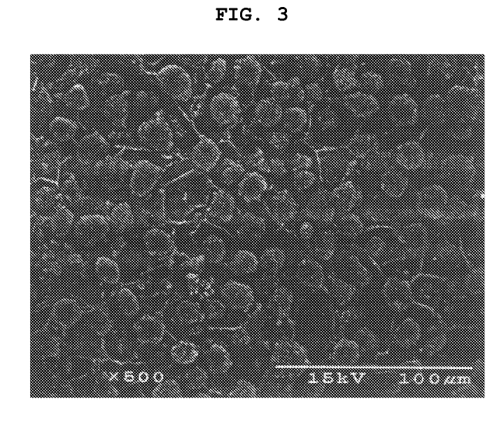

Porous chitosan beads of 1-4 mm with pores of 50-150 μm, prepared in Example 1, were neutralized with a 5 N sodium hydroxide/ethanol solution to remove remaining acids and organic solvents, followed by sterilization with 70% ethanol. After being applied to a culture medium (DMEM, pH 7.4, Gibco BRL, USA), the chitosan beads were freeze-dried. In a culture medium were immersed the freeze-dried chitosan beads to which hepatocytes from rats were then attached. For this attachment, preculturing was conducted for 4-6 hours. In order to remove the cells remaining unattached, the medium was changed with a fresh one. Since then, the medium was changed every two or three days for 1-10 days while the hepatocytes attached to the chitosan beads were cultured at 37° C. While being agglomerated, the cells were observed to grow in pores of the chitosan beads as well as over surfaces of chitosan beads, under a scanning electron microscope, as shown in FIG. 3.

### Experimental Example 2

### Culture of NIH3T3 Cells

Using NIH3T3 cells, which are fibroblastic cells (ATCC HB-11601, USA), the same procedure as in Experimental Example 1 was conducted for cell culture.

Observation under a scanning electron microscope revealed that the cells were firmly attached to the chitosan beads, as well as growing in the pores. Additionally, the fibroblastic cells were observed to rapidly grow and stably contacted to each other.

### Experimental Example 3

### Culture of MC3T3-E1 Cells

Using MC3T3-E1 cells, which are osteoblastic cells (Korean Cell Line Bank in Seoul National University College of Medicine, Seoul, Korea), the same procedure as in Experimental Example 1 was conducted for cell culture.

Under a scanning electron microscope, these cells were observed to stably attached to the chitosan beads and grow well.

### Experimental Example 4

### Culture of CHO-K1 Cells

Using CHO-K1 cells, which are epithelial cells (ATCC CCL-61, USA), the same procedure as in Experimental Example 1 was conducted for cell culture.

Under a scanning electron microscope, these cells were observed to firmly attached to the chitosan beads, as well as growing in the pores.

### Experimental Example 5

### Culture of PT67 Cells

Using PT67 cells, which are packaging cells (Korean Cell Line Bank in Seoul National University College of Medicine, Seoul, Korea), the same procedure as in Experimental Example 1 was conducted for cell culture.

Under a scanning electron microscope, these cells were observed to not only firmly attached to the chitosan beads while secreting extracellular matrix in a large quantity, but also rapidly grow.

### INDUSTRIAL APPLICABILITY

As described hereinbefore, the porous chitosan beads of the present invention have uniform pores thereon and therein such that they can be useful as matrices which provide three-dimensional structures useful in aiding cells to perform their functions. Additionally, over conventional matrices for cell culture, the porous chitosan beads of the present invention attain superiority in ability of cell attachment, biocompatibility, and biodegradability as well as in terms of cell growth, angiogenesis and nutrient diffusion. With these advantages, the porous chitosan beads of the present invention are useful as matrices for culturing animal and plant cells. Further to these, the porous chitosan beads can be effectively used for research on substitutes for metabolic tissues such as the liver and the pancreas, or cartilage or bones, as well as on the production of biologically useful materials, including proteins, antibiotics, anti-cancer materials, polysaccharides, biologically active materials, and animal and plant hormones.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1-3. (canceled)

4. A method for preparing porous chitosan beads, comprising:
providing a chitosan solution in which chitosan is dissolved in an aqueous acetic acid, an aqueous chitosan solution in which water soluble chitosan is dissolved in deionized water or a mixture thereof;
dropwise dropping the chitosan solution, the aqueous solution or mixture thereof into an organic solvent at a temperature of −5 to −65° C. to give beads; and
freeze-drying the chitosan beads.

5. The method as set forth in claim 4, wherein the chitosan has an average molecular weight of 30,000 to 100,000 daltons and the aqueous chitosan has an average molecular weight of 100,000 to 400,000 daltons.

6. The method as set forth in claim 4, wherein the aqueous acetic acid has a concentration of 1.0-4.0 wt %.

7. The method as set forth in claim 4, wherein the chitosan solution has a chitosan concentration of 0.5-2.0 wt %.

8. The method as set forth in claim 4, wherein the aqueous chitosan solution has a chitosan concentration of 0.5-1.54 wt %.

9. The method as set forth in claim 4, wherein the mixture has a weight ratio of the chitosan solution to the aqueous chitosan solution ranging from 2:8 to 8:2.

10. The method as set forth in claim 4, wherein the organic solvent is chlorocyclohexane, chloropentane, n-hexane, dichloromethane, chloroform or ethyl acetate.

11. The method as set forth in claim 4, wherein the organic solvent is maintained at −5 to −65° C.

12. The method as set forth in claim 11, wherein the organic solvent is chilled by use of ethanol maintained at −5 to −65° C. with the aid of dry ice or a freezer.

13. A method for culturing animal cells or plant cells, comprising:
providing a chitosan solution in which chitosan is dissolved in an aqueous acetic acid, an aqueous chitosan in which water soluble chitosan is dissolved in deionized water or a mixture thereof;
dropwise dropping the chitosan solution, the aqueous solution or mixture thereof into organic solvent at a temperature of −5 to −65° C. to give beads;
freeze-drying the chitosan beads;
neutralizing the porous beads to remove acids and organic solvents, followed by sterilizing the porous beads;
subjecting the porous chitosan beads to preculturing for 4-6 hours to attach the cells to the porous chitosan beads; and
refreshing a culture medium of cells attached to the chitosan beads, periodically.

14-15. (canceled)

16. A method for making animal or plant tissue, comprising:
providing a chitosan solution in which chitosan is dissolved in an aqueous acetic acid, an aqueous chitosan in which water soluble chitosan is dissolved in deionized water or a mixture thereof;
dropwise dropping the chitosan solution, the aqueous solution or mixture thereof into organic solvent at a temperature of −5 to −65° C. to give beads;
freeze-drying the chitosan beads;
neutralizing the porous beads to remove acids and organic solvents, followed by sterilizing the porous beads;
subjecting the porous chitosan beads to preculturing for 4-6 hours to attach the cells to the porous chitosan beads; and
refreshing a culture medium of cells attached to the chitosan beads, periodically.

17. The method according to claim 16, wherein said tissue is a metabolic tissue.

18. The method according to claim 16, wherein said tissue is liver, pancreas, cartilages or bone.

19. A method of producing biologically active material comprising:
providing a chitosan solution in which chitosan is dissolved in an aqueous acetic acid, an aqueous chitosan in which water soluble chitosan is dissolved in deionized water or a mixture thereof;
dropwise dropping the chitosan solution, the aqueous solution or mixture thereof into organic solvent at a temperature of −5 to −65° C. to give beads;
freeze-drying the chitosan beads;
neutralizing the porous beads to remove acids and organic solvents, followed by sterilizing the porous beads;
subjecting the porous chitosan beads to preculturing for 4-6 hours to attach the cells to the porous chitosan beads;
refreshing a culture medium of cells attached to the chitosan beads, periodically; and
inducing production of the biologically active material from the cells.

20. The method according to claim 19, wherein said biologically active material is a protein, antibiotic, anti-cancer material, polysaccharide or hormone.
